(19) United States
(12) Patent Application Publication
(10) Pub. No.: US 2023/0086646 A1
(43) Pub. Date: Mar. 23, 2023

(54) Therapeutic Eyewear Sleeve

(71) Applicant: Ronald Jerome McCoy, Tallahassee, FL (US)

(72) Inventor: Ronald Jerome McCoy, Tallahassee, FL (US)

(21) Appl. No.: 17/768,904

(22) PCT Filed: Jan. 14, 2021
(86) PCT No.: PCT/US2020/060812

§ 371 Date: Apr. 14, 2022

(51) Int. Cl. A61M 35/00; G02C 5/00

(52) U.S. Cl. CPC A61M 35/10; G02C 5/001

(57) ABSTRACT

Therapeutic Eyewear patent introduces new functionality for the eyewear frame (prescription frames, sunglasses, goggles) —the delivery of CBD or other therapeutic medicine via infused pads located anywhere on the frame, primarily those parts of the frame in contact with the wearer's skin.

I propose to use the eyewear (prescription frames, sunglasses, goggles) frame for the delivery of cannabidiol (CBD) or any other therapeutic medicine via infused pads to the wearer while the person is wearing the eyewear. The location of the delivery may be from anywhere on the frame, but primarily from areas in contact with the skin while the eyewear is being worn. The infused pads will be replaceable.

### CROSS REFERENCE TO RELATED APPLICATIONS

N/A

### FEDERAL RESEARCH OR DEVELOPMENT

N/A

### REFERENCE TO SEQUENCE LISTING

N/A

### BACKGROUND

Eyewear is utilized by a large segment of the world population to assist them in vision correction. It is also used to protect their eyes from the sun's harmful UV radiation, Since wearing eyewear is something that many people do on an everyday basis, this Therapeutic Eyewear patent will give them a passive method to get relief from CBD or any other therapeutic medicine that they may require.

### SUMMARY

This patent is a new use for eyewear. In addition to assisting wearers with vision correction and protection from UV radiation, I propose to now use the eyewear frame to deliver CBD or other therapeutic medicine to the wearer via replaceable infused pads positioned anywhere on the frame, primarily those parts of the eyewear frame that touch the skin to ensure the most efficient delivery of CBD or any other therapeutic medication.

### DESCRIPTION OF VIEWS

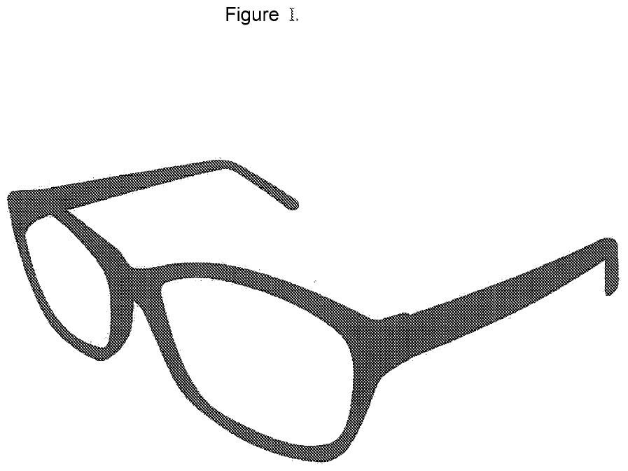

FIG. 1 depicts an eyewear frame that represents either prescription eyewear frames or sunglasses.

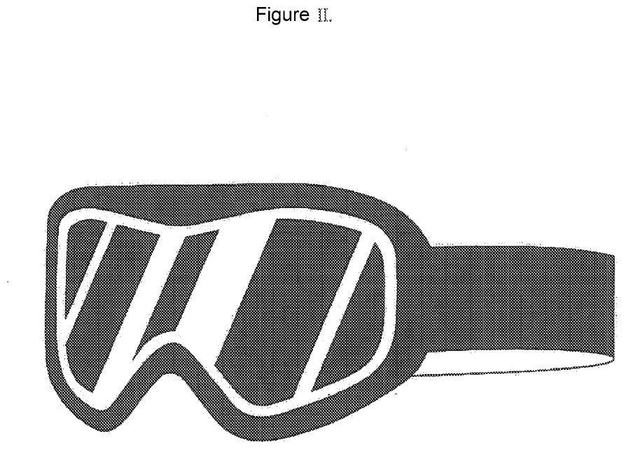

FIG. 2 depicts a pair of goggles with a rubber head band,

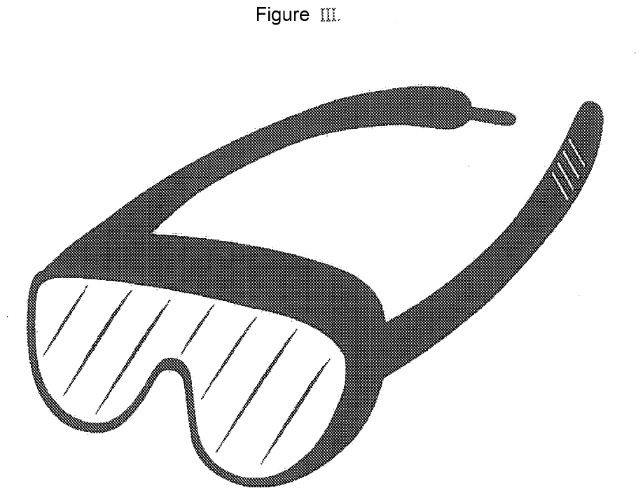

FIG. 3 depicts a pair of goggles with temple arms.

### DETAILED DESCRIPTION

Therapeutic Eyewear introduces a new function for eyewear, which is a delivery of therapeutic relief from pads infused with either CBI) or other medicine to be absorbed into the skin of the person while wearing the eyewear.

Up until now, eyewear has principally been used all over the world for corrective vision purposes, protection from the sun's ultraviolet radiation, and/or protection from dangerous chemicals or other objects entering the eyes.

The therapeutic infused pads could be placed anywhere on the eyewear frame, but primarily in places where the eyewear is in contact with the skin. The therapeutic infused pads would be replaceable.

The therapeutic infused pads would be inserted into slots located at various places on the eyewear frame such as: 1) inside arm of frame near ear area, 2) underside of temple arm that rests on the ear, and 3) on both sides of the nose area.

Due to the multitude of ways that eyewear is manufactured, the attached drawings depict three of the most popular styles of eyewear. The intent of this Therapeutic Eyewear patent is to include all styles of eyewear regardless of how it is made to remain securely in place on the head of the wearer.

1. I claim new functionality for eyewear frames (prescription frames, sunglasses, goggles) by modifying the frame to house pads infused with CBD or other therapeutic, medicine which would be absorbed into the skin while wearing the eyewear.
